(19) United States
(12) Patent Application Publication
(10) Pub. No.: US 2014/0142407 A1
(43) Pub. Date: May 22, 2014

(54) ELECTRODES AND METHODS OF USE

(71) Applicant: The Salk Institute for Biological Studies, La Jolla, CA (US)

(72) Inventors: Anita Disney, San Diego, CA (US); Jude Mitchell, La Jolla, CA (US)

(21) Appl. No.: 14/074,413

(22) Filed: Nov. 7, 2013

Related U.S. Application Data
(60) Provisional application No. 61/727,599, filed on Nov. 16, 2012.

(51) Int. Cl. A61B 5/00

(52) U.S. Cl. CPC A61B 5/6868
USPC 600/378; 600/372; 29/874

(57) ABSTRACT

Disclosed are methods and devices for simultaneous recordings of neuronal electrical activity and their immediate chemical environment on subsecond timescales. Due to its sub-300 micron size, the device can be used in chronic recordings in higher mammals (particularly primates) with minimal resulting tissue damage, allowing studies of the relationship between brain chemistry, neuronal activity and behavior in complex tasks as they evolve over time.

### CROSS REFERENCE TO RELATED APPLICATION

This application claims the benefit of U.S. Provisional Patent Application No. 61/727,599, filed Nov. 16, 2012, which application is hereby incorporated by reference in its entirety.

### ACKNOWLEDGMENT OF GOVERNMENT SUPPORT

This invention was made with government support under Grant No. MH 093567 awarded by National Institutes of Health. The government has certain rights in the invention.

### FIELD

This disclosure relates to the field of electrodes, such as electrodes used for electrochemical and electrophysiological recordings in the brains of higher mammals.

### BACKGROUND

Brain function has been studied for many years by measuring brain electrical and/or chemical activity. Local field potentials (LFPs) are a class of electrophysiological signals, which includes the electrical current flowing from all nearby synaptic activity within a volume of tissue. Recent studies have suggested that LFP abnormalities can be identified in a number of neurological disorders. Local field potentials have also been used in the development of successful neural prostheses (a series of devices that can substitute a motor, sensory or cognitive modality that might have been damaged as a result of an injury or a disease).

An alternate electrophysiological signal that can be measured is single unit activity (SUA), which includes the current flowing across the membrane of a single neuron's cell body. Recording single unit activity is the only way to determine how individual neurons operate within cortical circuits.

To further elucidate the role of LFPs and SUA in brain processes, including in neurological disorders and neural prostheses, methods and devices are needed that allow for simultaneous assessment of extracellular neurochemical concentrations and LFPs and SUAs. The presently available methods and devices fail to meet this need. For example, some methods utilize a single electrode operating in series mode alternating from recording electrochemical and electrophysiological signals. This allows electrochemical and electrophysiological signals to be recorded at the same location, but not at the same time. Alternative systems utilize two electrodes operating in parallel, allowing simultaneous electrochemical and electrophysiological recording, but not at the same location. A third system allows concurrent LFP below 20 Hz and electrochemical recording, but not recording of LFPs in the 20-200 Hz range (frequencies that contain important signals such as gamma band synchronization at 30-80 Hz which has been implicated in conscious awareness) or isolation of SUA.

### SUMMARY

Disclosed herein are methods and devices for simultaneous recordings of the neuronal electrical activity (full frequency range LFP and/or SUA) and their immediate chemical environment on rapid timescales. In particular embodiments, the disclosed methods make simultaneous (sub-second timescale) recordings of the electrical activity of neurons and their co-located (sub-millimeter location scale) chemical environment with high (sub-second) temporal resolution. For example, the inventors have demonstrated that a disclosed electrode can be used in chronic (repeated) recordings in higher mammals (particularly primates) with minimal resulting tissue damage, thereby allowing the relationship between brain chemistry, neuronal activity and behavior in complex tasks to be studied as they evolve over time.

Disclosed herein is an electrode capable of simultaneously recording neuronal electrical activity and the co-located chemical environment with high (sub-second) temporal resolution. In some embodiments, a disclosed electrode includes a first electrical circuit enabling electrophysiology recording and a second electrical circuit enabling electrochemical recording. In some embodiments, a disclosed electrode is capable of piercing thickened primate dura. In some embodiments, a disclosed electrode is coupled to a hypodermic tube, such as a hypodermic tube with an outer diameter less than or equal to 0.6 mm and/or at least 1 cm in length. In some embodiments, the electrode has a width less than or equal to 0.4 mm. In some embodiments, the electrode is compatible with guide systems used in primate recording. In some embodiments, the electrode can be inserted to any depth within the primate brain.

Also disclosed herein are methods of making an electrode, such as a mounted electrode for simultaneously recording electrophysiological and electrochemical activity. In some embodiments, the method includes creating one or more circuits on a wafer; dicing the wafer to isolate an electrode; and combining the electrode and a hypodermic tube. In some embodiments, the electrode and the hypodermic tube together are capable of piercing thickened dura of a primate brain and inserting the electrode into the primate brain. In some embodiments, the hypodermic tube has an outer diameter less than or equal to 0.6 mm. In some embodiments, the electrode has a width less than or equal to 0.4 mm. In some embodiments, the electrode is compatible with guide systems used in primate recording. In some embodiments, the electrode can be inserted to any depth within the primate brain. In some embodiments, the hypodermic tube is at least 1 cm long.

The foregoing and other features of the disclosure will become more apparent from the following detailed description, which proceeds with reference to the accompanying figures.

### BRIEF DESCRIPTION OF THE DRAWINGS

FIG. 1 is a schematic of an exemplary electrode illustrating a plurality of electrical circuits arranged within the electrode;

FIG. 2 is a drawing of an exemplary first layer photolithography mask;

FIG. 3 is a drawing of an exemplary second layer photolithography mask;

FIG. 4 is a schematic of scribe lines and electrical circuits onboard a disclosed electrode;

FIGS. 5A-5C are drawings illustrating the dimensions and taper angle of a disclosed electrode;

FIG. 6 is a schematic drawing of cut lines used in dicing wafers;

FIG. 7 is a schematic showing the dimensions of an exemplary electrode;

FIG. 8 is a pair of digital images of an exemplary bonder stage adapter used with fixed-width electrodes;

FIG. 9 is a digital image of an exemplary bonder stage adapter used with variable-width electrodes of the methods and devices disclosed herein;

FIG. 10 is a set of digital images of an exemplary electrode attached to a hypodermic tube in a non-dura-piercing configuration of the methods and devices disclosed herein;

FIG. 11 is a set of digital images of an exemplary electrode attached to a hypodermic tube in a dura-piercing configuration of the methods and devices disclosed herein;

FIG. 12 is a pair of digital images showing a mounted electrode in a dura-piercing configuration for use with the methods and devices disclosed herein;

FIG. 13 is a schematic drawing illustrating acrylic slug; and

FIG. 14 includes an electrophysiological tracing and an electrochemical tracing acquired with an exemplary electrode positioned in the primary visual cortex.

### DETAILED DESCRIPTION

### I. Introduction

An electrode is an electrical device capable of forming one or more independent electrical connections between two locations, typically between a piece of equipment whose accurate location is not precisely critical and a volume or structure within a biological specimen where the accurate location of that volume or structure is relevant. In some examples, an electrode bridges between a macroscopic location (such as the terminal on a piece of recording equipment or the macroscopic electrical lead wires attached to a recording device) and a microscopic location such as is found within a biological specimen and which microscopic location can be specified to within less than a centimeter or less than a millimeter. An electrode can include one or more electrical circuits which can create an electrical path between the two locations above. An electrode can include one or more additional electrical circuits designed to facilitate or improve forming the above connection and performing accurate recording. These electrical circuits can include various electrical components, for example resistors, capacitors, inductors, diodes, transistors, amplifiers, and other components known to those of skill in the art.

An electrical recording device or a piece of electrical recording equipment enables the act of recording which is to accurately measure and store and/or retain one or more values for a physical property of a specimen, for example an electrical current, a voltage, a resistance value, a capacitance, or the concentration of a chemical or a molecule.

The methods and device disclosed herein can be used to make simultaneous recordings of the electrical activity of neurons and their immediate chemical environment on sub-second timescales. These methods of construction and the resulting devices can be used in chronic recordings in higher mammals (particularly primates) with minimal resulting tissue damage, allowing studies of the relationship between brain chemistry, neuronal activity and behavior in complex tasks as they evolve over time.

Simultaneous as used herein means that recordings of two or more related signals or properties of a specimen, e.g., electrical and chemical properties, are recorded closely enough in time that any time difference or lag is insignificant relative to the rates of change in the signals being recorded. Simultaneous can mean a lag of less than a minute, or less than a second, or less than a millisecond. Given rates of change of over 1% per millisecond as are typical in electrochemistry and physiology, a lag between recordings of less than a millisecond is preferable. This disclosure facilitates essentially concurrent or exactly simultaneous recordings using parallel recording channels so that lag approaches zero.

Methods are taught herein for fabricating electrodes using photolithography techniques and for mounting electrodes into “mounted electrode assemblies,” or simply “mounted electrodes.”

### II. Electrodes and Mounted Electrode Assemblies

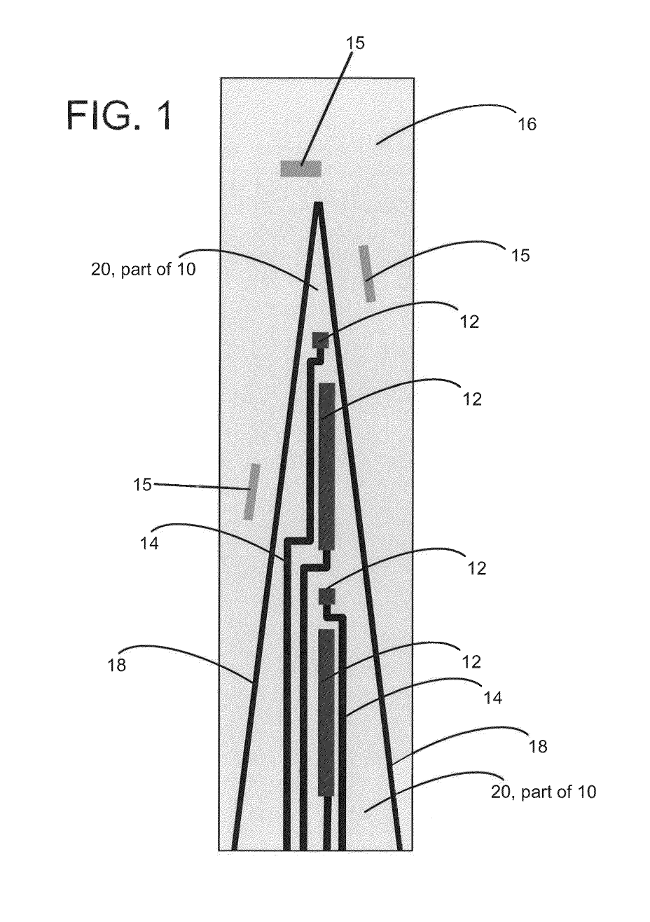

With reference to FIG. 1, a partial view of an exemplary electrode 10 is provided. FIG. 1 illustrates the electrode tip 20 of the complete electrode 10 after the electrode is diced from a substrate or wafer 16 along cut lines 18 (as described in detail below, see Section III). Scribe lines 15 are shown indicating the position that a cutting device, such as a knife, of known blade width is centered to cut each individual tip out of the wafer 16.

In order to record using electrode 10 within the brain, one or more electrical contacts 12 are fabricated at the ends of one or more electrical circuits (referred to as connecting lines 14). In some embodiments, electrical contacts 12 are fabricated via photolithography as described below (see, for example, Section III). For example, photolithography can be used to fabricate electrodes with circuits enabling combined simultaneous amperometry and physiology.

In some embodiments, electrodes are diced to widths of 1 cm and smaller, such as 1 mm and smaller or 300 microns and smaller. For example, the width of an electrode after dicing is from about 0.004 to about 0.2 inches (approximately 0.1 to 5.0 mm), such as between about 0.012 to about 0.02 inches (approximately 0.3 to 0.5 mm), including 0.010 inches, 0.011 inches, 0.012 inches, 0.013 inches, 0.014 inches, 0.015 inches, 0.016 inches, 0.017 inches, 0.018 inches, 0.019 inches or 0.020 inches.

In some embodiments, a disclosed electrode is mounted into hypodermic tubing to fabricate a mounted electrode assembly. In some specific embodiments, a mounted electrode has a maximum width (outer diameter) of 1 mm or smaller, such as 0.6 mm or smaller, for example, 0.415 mm (non-during-piercing hypodermic configuration) or 0.565 mm (dura-piercing hypodermic configuration). In some examples, hypodermic tubing ranges between inner diameters of 0.004 inches to 0.05 inches (roughly between 0.1 and 1.27 millimeter (mm)) and outer diameters of 0.005 inches to 0.06 inches (roughly between 0.12 and 1.5 mm). In some examples, tubing gauge ranges from 18 to 38, such as from 20 to 28 or 23 and 24.

In some examples, a mounted electrode (electrode and hypodermic tubing) has a total combined length of greater than 1 cm, including greater than 2 cm, greater than 3 cm, greater than 4 cm, greater than 5 cm, or greater than 6 cm, while retaining desirable rigidity and strength. It is contemplated that tubing length can range from 0.5 inch to 24 inches (roughly between 1.0 and 20 centimeters (cm)) such as from 6 inches to 14 inches (roughly between 15 and 35 cm), including 6 inches, 7 inches, 8 inches, 9 inches, 10 inches, 11 inches, 12 inches, 13 inches, 14 inches etc.

### III. Methods of Fabricating Electrodes

Disclosed are methods of making electrodes, such as a mounted electrode, for simultaneously recording electrophysiological and electrochemical activity. In some embodiments, the method includes creating one or more circuits on a wafer; dicing the wafer to isolate an electrode; and combining the electrode and a hypodermic tube. It is contemplated that any of these steps described herein (unless otherwise indicated) can be repeated and/or omitted. Further, the order of performing steps can also be varied.

A. Selection and Preparation of a Clean Wafer for Photolithography

The wafer used for the methods herein can be any wafer meeting generally understood standards for photolithography. In particular, any thin ceramic substrate or silicon substrate/wafer may be used. For simplicity, the steps below refer to processing a single wafer. In an example one or more wafers may be processed. If more than one wafer is processed, then one or more steps below may be performed in series or in parallel for each wafer.

A selected wafer can be prepared for use herein by cleaning according to methods known to those of ordinary skill in the art. In some examples, a selected wafer is prepared by cleaning according to the following steps:
i. Place wafer in a vessel.
ii. Bathe for about 5-20 minutes (preferably 10 minutes) in pure acetone while applying ultrasonic agitation.
iii. Bathe for about 5-20 minutes (preferably 10 minutes) in pure methanol.
iv. Bathe for about 5-20 minutes (preferably 10 minutes) in pure isopropanol.
v. Bathe for about 5-20 minutes (preferably 10 minutes) in de-ionized water.
vi. Expose a wafer to a stream of Nitrogen gas, for example using a Nitrogen gun, until wafer is dry.

B. Photolithography

In some examples, photolithography is used to fabricate electrical contacts within a disclosed electrode. For example, the following photolithography steps (1-6) are repeated for a first layer using a first layer photolithography mask and then a second layer, using a second layer photolithography mask. In other embodiments, these steps can be repeated for additional layers using additional masks.
1. Selection and Application of Photoresist to a Layer

The photoresist used for the methods herein can be any photoresist known to one of ordinary skill in the art (such as a Shipley S1813 photoresist for the first layer, and Microchem SU-8 2001 for the second layer). The desired thickness of the metal of the final electrode can be a criteria used to select first layer photoresist. Electrical insulation properties and biocompatibility can be criteria used to select the second layer resist. The selected photoresist can be applied according to the manufacturer's directions and according to methods known to those of ordinary skill in the art.
i. Bring a heat source to about 160-200° C. (preferably 180° C.).
ii. Place a wafer on the heat source and allow the wafer to dry for about 5-20 minutes (preferably 10 minutes).
iii. Allow to cool (e.g., reach room temperature, such as 15-35° C.)
iv. Select an attachment for use with a spinner (i.e. a spinner chuck), and clean the spinner chuck with acetone.
v. Deposit photoresist (e.g. S1813) onto the wafer in a steady stream (without spinning) vertically above wafer center.
vi. Spin/bake according to manufacturer recommendation for selected resist. For example, for a S1800 series resist: provided below are the spinning and baking parameters particular to the S1800 series resists. For example, starting with an acceleration of about 150-900 rpm (revolutions per minute), spin the wafer at about 4,500-5,000 rpm for about 45-90 seconds. This step may be repeated as Spin 1, Spin 2, etc., and these operating parameters can be varied, as described below.
vii. Transfer wafer to a heat source with a surface temperature about 90° C.-125° C. and pre-bake for 60-180 seconds.
viii. Determine if coating is adequate. If coating is insufficient or have surface imperfections, put back on spinner, apply vacuum and start spinning. Apply acetone to remove resist, rinse with water, blow off with N2 and repeat steps v-viii.

In some examples, the following parameter values are preferred for the respective layer and steps as described above:

Layer 1 of Photolithography Using Shipley S1813 Resist
Spin 1 of step vi: speed 4,500, time 45 seconds, acceleration 600
Pre-bake of step vii: temperature 115° C., time 1 minute

Layer 2 of Photolithography Using Microchem SU-8 2001 Resist
Spin 1 of step vi: speed 500, time 5 seconds, acceleration 150
Spin 2 of step vi: speed 3000, time 30 seconds, acceleration 400
Pre-bake of step vii: temperature 95° C., time 2 minutes
2. Exposing Photoresist Using the Mask for a Layer

Place mask for applicable layer in carrier and load into a contact aligner system (e.g. KarlSuss MA-6 Contact Aligner). Expose using the following exposure parameters preferably: for first layer ˜3-4 sec for ceramic or 6.5 sec for Si, and for second layer time=J/W where J is exposure time for selected SU-8 thickness, from product insert and W is the intensity of the mask aligner output. As an example, for a second layer of SU-8 2001, use 3-4 seconds exposure. Other exposure parameters can be the same, for example, for first and second layers: alignment gap: 80-120, contact: soft or hard, WEC type: Cont, WEC offset: +0, WEC pressure: 0.3-0.5 bar for ceramic or up to 0.7 bar for Si.

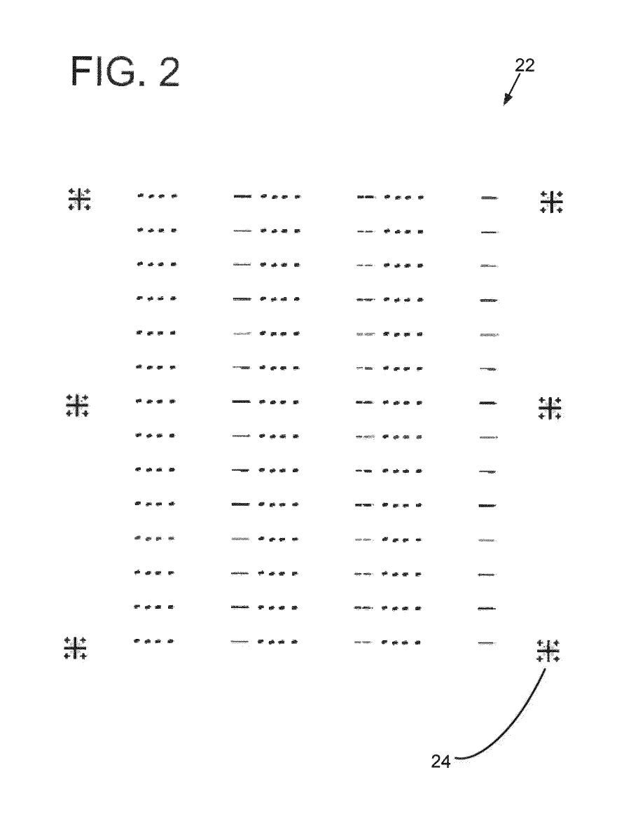

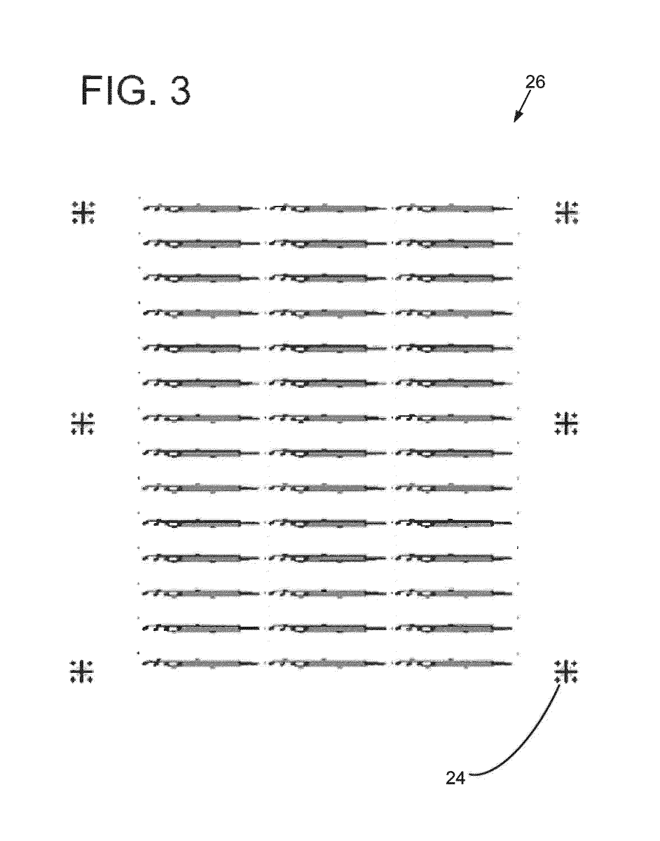

FIG. 3 shows an example of a first layer photolithography mask 22 used as explained in the steps above to fabricate multiple electrodes on a wafer. Also shown are alignment marks 24 used to ensure proper and repeatable alignment of the first mask with the wafer and with other masks. Similarly, FIG. 2 shows an example of a second layer photolithography mask 26 used as explained in the steps below.
3. Developing Photoresist

Photoresist can be developed by any method known to one of ordinary skill in the art. In some examples, a disclosed method includes developing a first layer of photoresist on a wafer followed by developing a second layer of photoresist.
First Layer Protocol

Using a developer and developing fluid, for example a Shipley MF 319, a layer of photoresist on a wafer can be developed in 30-180 (preferably about 90) seconds in Shipley MF 319 developing fluid. After developing, the wafer can be rinsed for 30-180 (preferably about 60) seconds in still de-ionized water. After this first rinse, the wafer can be rinsed for 30-180 (preferably about 60) seconds in running de-ionized water. Then both sides of the wafer can be exposed to a Nitrogen gas stream, e.g. by using a Nitrogen gun to remove water.
Second Layer Protocol

For the second layer, the photoresist can be post-expose baked at 75-100 (preferably about 95)° C. for 1-10, such as about 2, about 3, about 4, about 5, about 6, about 7, about 8, about 9 or about 10 minutes, for example, by using a heated surface (such as a hotplate). Using a developing fluid (e.g., SU-8 developing fluid by Microchem), a layer of photoresist on a wafer can be developed in 30-180 (preferably about 60) seconds, preferably, using the developing fluid in combination with ultrasonic agitation. After developing, the wafer can be rinsed for 2-60 (e.g., about 10) seconds in still SU-8 developing fluid. After this first rinse, the wafer can be rinsed for 30-180 (e.g., about 60) seconds in still isopropyl alcohol. Then both sides of the wafer can be exposed to a Nitrogen gas stream (e.g. by using a Nitrogen gun) to dry.

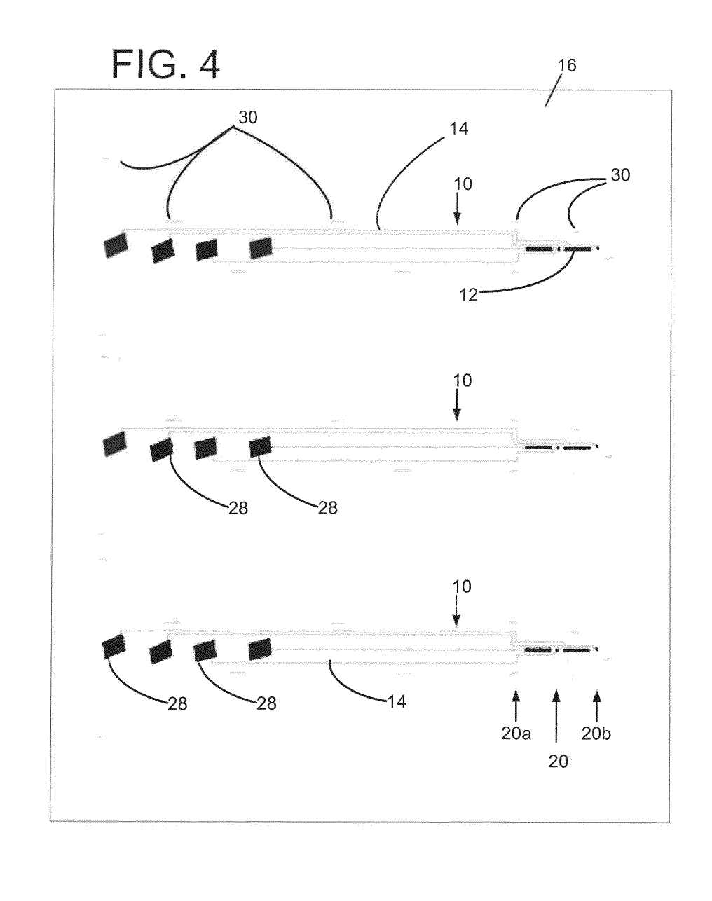

The recording sites 12 and bonding pads 28 of FIG. 4 can be inspected to ensure that sufficiently large openings in the insulation have been formed over these features to enable electrical connectivity. Openings should correspond to the size of the recording or bonding pad below and should leave a fully-exposed platinum surface. If openings are too small, these developing steps can be repeated, with reduced or increased durations as desirable.
4. De-Scumming a Layer

A photoresist layer on the wafer should be de-scummed after development. This can be achieved using an etching device, for example a Technics PE-11B Etcher. Under a vacuum of about 15-30 (e.g., about 20) milliTorr (mT), an Oxygen atmosphere can then be created at 180-220 (e.g., about 200) mT, and 150-250 (e.g., about 200) Watts (W) of power can be applied for 30-300 (preferably about 60 for the first layer and 180 for the second layer) seconds. Prolonged de-scumming times (>1 minute) can remove the photoresist layer and should be used with caution.
5. Sputter Depositing onto a Layer

Under Argon gas and with sample rotation, about 5-20 (e.g., about 10) nanometers (nm) Titanium (Ti) can be sputtered onto the wafer to create an adhesion layer. Then about 200-500 (preferably about 250) nm Platinum (Pt) can be sputtered onto the wafer to create the electrical paths of a layer. As an example, using the AJA ATC Orion DC Sputter Deposition System, process parameters for the Ti sputtering can be: 150-200 (e.g., about 200) Watt, 4-10 (preferably about 5) stnd cm3/min (sccm), 2-5 (e.g., about 3) milliTorr, 50-300 (e.g., about 135) seconds at a sample height of 20-40 (e.g., about 30) centimeters, sputter system specific. As an example, using the AJA ATC Orion DC Sputter Deposition System, process parameters for the Pt sputtering can be: 200-300 (e.g., about 300, 300 is max output of most systems) Watt, 4-10 (e.g., about 5) sccm, 2-5 (e.g., about 3) milliTorr, 5-20 (preferably about 7.5) minutes at sample height of 20-40 cm (e.g., about 30 cm).
6. Lifting-Off Residual Photoresist from a Layer

In consecutive steps of 1 to 30 minutes duration, the wafer can be immersed in acetone with ultrasonic agitation, immersed in methanol, immersed in isopropanol, and finally immersed in de-ionized water. Both sides of the wafer then can be exposed to a Nitrogen gas stream (e.g. by using a Nitrogen gun). In some examples, the duration of each step is about 10 minutes. Initial acetone immersion can be of durations up to 72 hours if photoresist is difficult to remove.

In some examples, to manufacture an electrode, the photolithographic layer is applied after sputtering on platinum. For example, a method a manufacturing includes cleaning a wafer; applying layer 1 resist; exposing layer 1 resist; developing layer 1 resist; descumming; sputtering Ti; sputtering Pt; lifting off residual photoresist; applying layer 2 resist; exposing layer 2 resist; developing layer 2 resist; descumming; and dicing (as detailed below).

C. Dicing a Wafer

As shown in FIG. 4, after photolithography, the wafer 16 has fabricated on it the miniature electrical circuits 14 of multiple electrodes 10 and a layer of electrical insulation (e.g. SU-8 or polyimide). The wafer 61 in FIG. 4 is rectangular and includes three electrodes 10, however, in some examples, a wafer is round or square and sufficient size (such as 1-4 inches) to fabricate dozens of electrodes. Various wafer sizes and geometries are possible.

In some examples, one or more electrical lead wires 29, which can electrically connect to recording equipment, is bonded to one or more bonding pads 28. When fabrication and assembly are complete, an electrode and its circuits can be used to bring recording equipment into electrical contact with target brain areas.

Since FIG. 4 shows each electrode 10 from its most distal end including bonding pads 28, to its most proximal end including its electrical contacts 12, the entire electrode is shown. The electrode tip 20 is that proximal portion of the electrode 10 roughly including the electrical contacts 12. In some embodiments, the width of the electrode tip 20 is roughly tapering from wider at its distal end 20a to narrower on its proximal end 20b. This width and tapering are discussed further below. The small squares shown in FIG. 4 are sites for recording electrophysiology data and the long rectangles are sites for recording electrochemistry data such that the “dashes” are electrochemistry the “dots” are electrophysiology.

In the present step, the electrodes 10 can be separated from the wafer 16 by cutting or dicing. Dicing can be accomplished using a cutting device, for example a dicer (e.g. a DISCO Automated Dicing Saw, DAD3220), and cutting the wafer 16 along cut lines (not shown) which follow scribe marks 30 as described below.

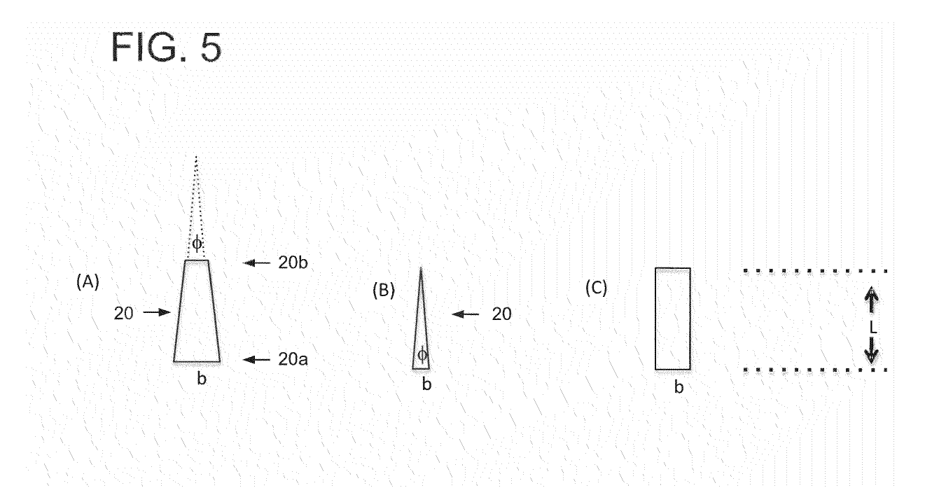

Regarding the taper of the electrode tip 20, FIG. 5 illustrates different possible embodiments. In some embodiments, the width b of the base of the electrode tip approximately equals the maximum width of the electrode. Thus, the width b refers equally to the entire electrode 10 and to the electrode tip 20. If there is any taper, the taper begins or is widest at this distal end of the electrode tip, at its base and tapers in width toward the proximal end of the electrode tip. The width b of an electrode can be from 0.004 to 0.2 inches (roughly 0.1 to 1.0 mm). In some examples, the electrode width ranges from 0.012 to 0.02 inches (roughly 0.3 to 0.5 mm). In some examples, the electrode width b is 0.016 inches (roughly 0.4 mm).

A taper angle φ is determined by the scribe marks and the angle at which an electrode is cut from a wafer. FIG. 5(A) shows an embodiment, in which a significant taper angle exists and the proximal end of the electrode tip 20b is cut to a blunt end. As shown in FIG. 5(B), in another example an electrode tip has a less blunt proximal end with the taper angle continuing approximately to the end. As shown in FIG. 5(C), in still another example, an electrode tip has a taper angle so that the entire electrode tip is approximately rectangular. The angle φ can be as little as 1 degree, resulting in an essentially triangular electrode tip as shown in FIG. 5(B). Alternatively, the angle φ can be as much as 90 degrees, resulting in an essentially rectangular electrode tip. In a specific embodiment, φ is 10-20 degrees. In another specific embodiment, φ is 5.5 degrees. L is the length from the distal end 20a of an electrode tip to the proximal end 20b.

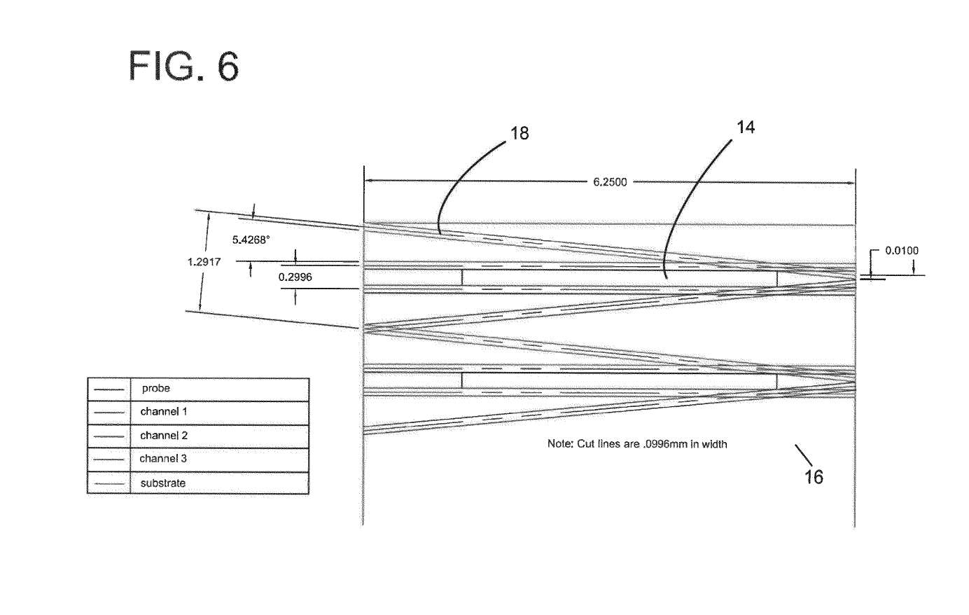

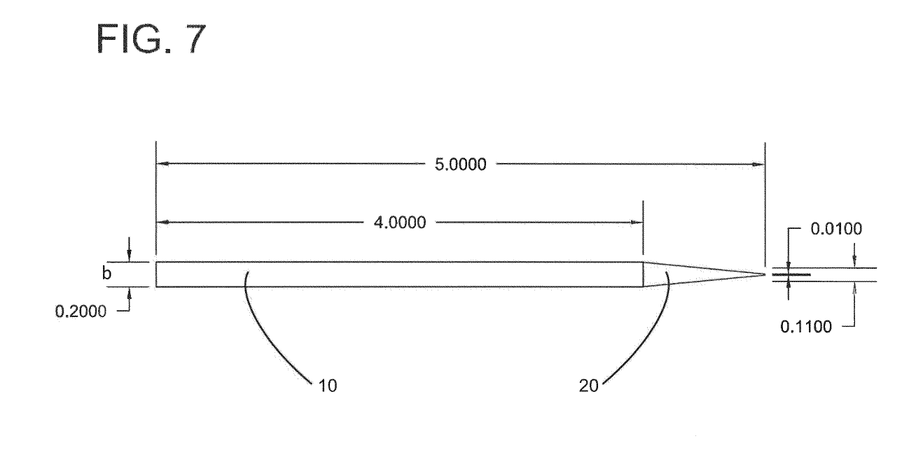

FIG. 6 shows an example of multiple cut lines 18 used to dice an electrode 14 from a wafer. The cut lines 18 can follow or be aligned with scribe marks 30 (see FIG. 4). FIG. 7 shows the dimensions in millimeters of an example electrode 10 and its electrode tip 20 after dicing. The electrode shown has a width b of 0.2 millimeters (200 microns). The length of the entire electrode is 5.0 millimeters. The length less the electrode tip is 4.0 millimeters. Thus, the length of the electrode tip in the present example is 1.0 millimeter. The width of the end of the electrode tip is 0.01 millimeter (10 micron).

As an example dicing protocol, wafers are cut or diced into electrodes as follows. A wafer is mounted so as to hold the wafer stationary before and during dicing and to hold the electrodes stationary after dicing. For example, UV release tape is used for mounting during dicing, and UV light is then used to release electrodes after dicing. As an example, dicing is accomplished using a DISCO Automated Dicing Saw (e.g. DAD3220).

As an example, dicing includes use of 10 to 30 micron electroformed dicing blades operated at blade translation speeds of 0.1-1 mm/s. For the fine tip, speeds below 0.2 mm/second are needed, for the ceramic substrate in general, speeds up to 1 mm/second can be tolerated for straight cuts.

### IV. Assembling a Mounted Electrode

Methods of assembling a mounted electrode are also disclosed. It is contemplated that any of these steps described herein (unless otherwise indicated) can be repeated and/or omitted. Further, the order of performing steps can also be varied.

A. Wire Bonding

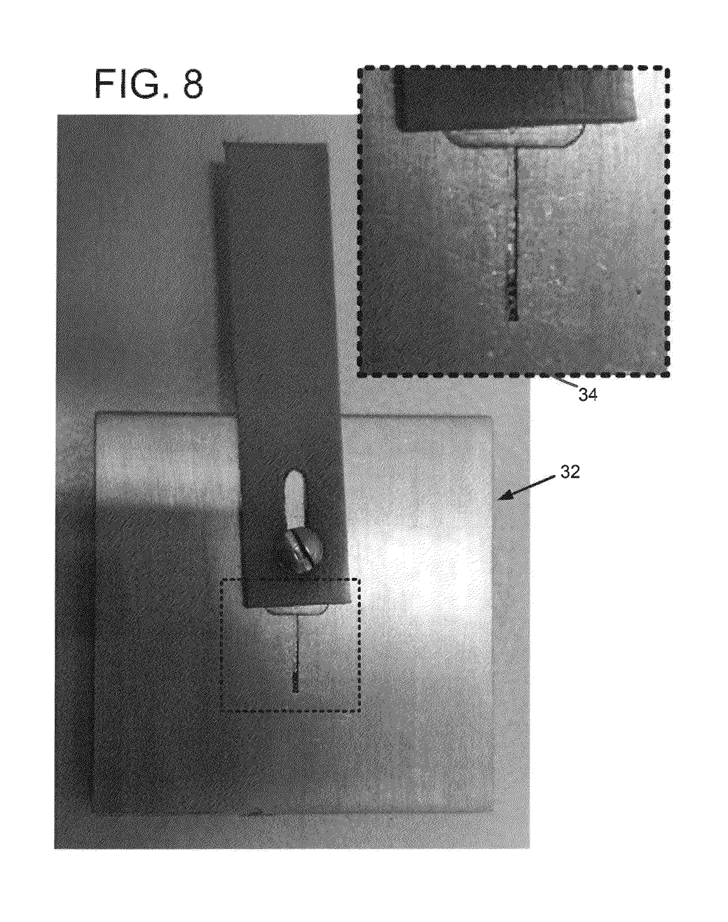

Before bonding electrical lead wires to the bonding pads 28 (FIG. 4) of an electrode 10, the electrode can be stabilized. FIG. 8 shows an example of a bonder stage adapter 32. A bonder stage adapter 32 can be fit over a standard bonder stage (not shown) and can fluidly connect the vacuum of the standard bonder stage to holes 34 (see inset) in the bonder stage adapter 32, thereby positioning that vacuum source to hold an electrode 10 (FIG. 4). A bonder stage adapter can be desirable because of the smaller electrodes of the present disclosure in comparison to those used with a standard bonder stage. Thus, a bonder stage adapter can be used to hold an electrode 10 of the present disclosure while electrical lead wires are bonded to the bonding pads 28 (FIG. 4) of the electrode.

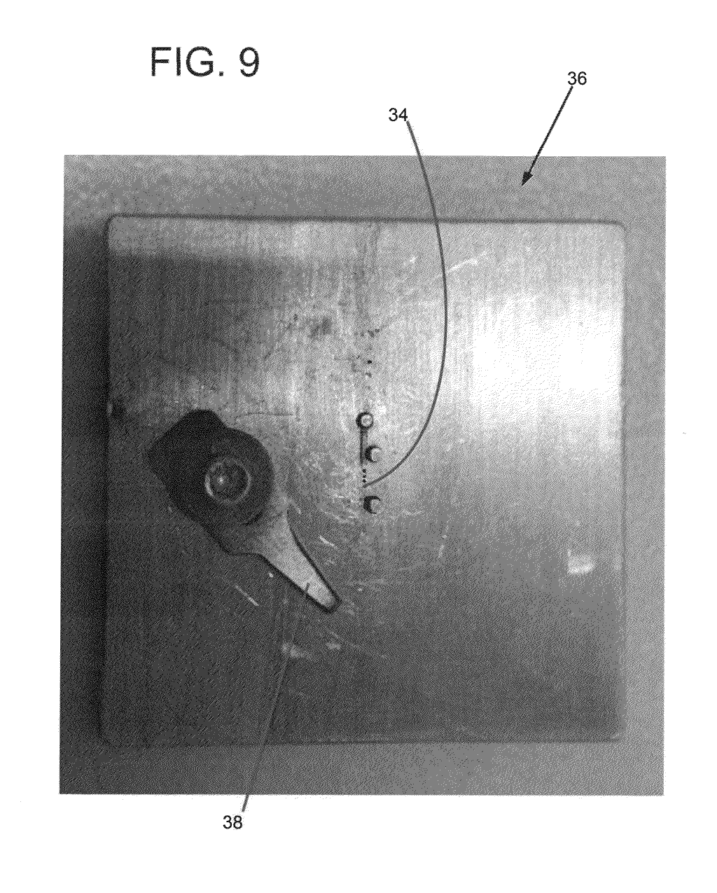

FIG. 9 shows another example of a bonder stage adapter 36, this one suitable for holding variable width electrodes because it uses an adjustable arm 38 to hold an electrode.

As an alternative to using a vacuum bonder stage adapter, an adhesive (e.g., such as a cyanoacrylate, including methyl-2-cyanoacrylate, ethyl-2-cyanoacrylate, n-butyl cyanoacrylate, 2-octyl cyanoacrylate or any combination thereof) can be used to hold an electrode to a glass slide. The electrode is adhered to the glass slide, the electrical lead wires are bonded to the bonding pads, and then the electrode is freed from the glass by exposing the superglue to acetone.

With an electrode stabilized, the following steps are an example protocol for bonding:

98% pure platinum (Pt) wire (such 99% or more pure, and most preferably Pt wire is 99.9%) with 1× natural polyimide insulation can be selected. In some examples, gold wire is utilized. In other examples, any biocompatible insulation may be utilized with modifications to the bonding protocol below. A West:Bond 7476E bonding tool can be reconfigured for 5 Watt output (in contrast to its default 2.5 Watt output). Using the bonding tool, the main spring and counterweight can be removed so that the force applied is the full dead weight of the tool (approximately 380 g). A bonding tool can be used which is configured to the bonding pad, wire diameter and polyimide thickness. For example, the following tools can be used:

MF-108-1/16-750-0.006-0.007-M-N-2.4 TDF=0.040, GD0.001 (Deweyl) or

MF-108-1/16-750-0.008-0.008-M-N-2, TDF=0.040, GD0.0008 (Deweyl) (M refers to a tool suited for ceramic; F-108 is a line of bonding tools suited to insulated wire; 1/16 refers to 1/16 inches tool diameter; 750 refers to tool length, 0.75 inches. 0.008-0.008 refers to the “foot size” of the tool (the foot being the part of the tool that comes into contact with the wire and electrode, 0.008×0.008 or above 0.006×0.007 refers to the dimensions of this foot in inches); M is matte finish; N refers to the foot radius option in which the left and right halves of the foot are the same size; 2 refers to the groove depth).

The bonder settings tool can be as follows:

Power: 850 (750-900 work but reliable bonding at 850)

Time: 275 (225-300 work but reliable bonding at 275)

Stage: 100° C. (stage can be unheated)

Tool: 100° C.

One Pt wire can be bonded to each bonding pad

In some examples, one wire, such as one Pt wire, is bonded to a bonding pad. In some examples, each wire is labeled or fashioned to indicate whether it is connected to a physiology or amperometry channel.

B. Preparation of a Mounting Assembly Including an Electrode and a Hypodermic Tube

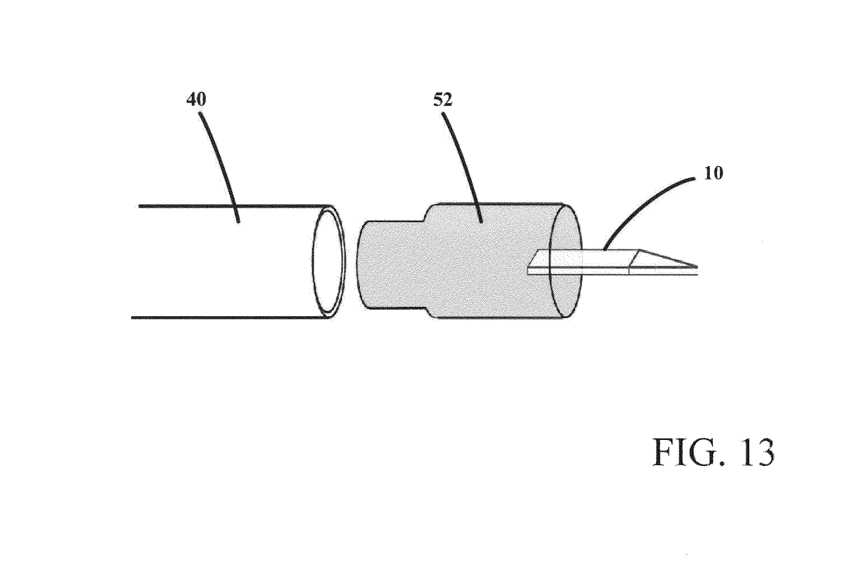

As an example, electrodes can be assembled into a section of hypodermic tubing called a hypodermic tube to fabricate a mounting assembly or mounted electrode. An alternative configuration would, for example, use molded dental acrylic or SU-8 photoresist in the place of part or all of the tubing to encapsulate the wires and provide protection, rigidity and desired length to this device. The use of dental acrylic can allow fabrication of devices <0.3 mm in diameter while preserving desired strength and rigidity and also provides desirable additional electrical insulation over the bonding pads with bonded Pt wires attached. In one example, the acrylic or resist is molded so as to cover the distal 3 mm of the electrode and the proximal 2.3 cm of the insulated Pt wires. The distal end of the acrylic is molded so as to fit inside the lumen of the selected hypodermic tubing. Thus the acrylic provides a 2.5 cm insulated, strong, small diameter “bridge” between the electrode and the hypodermic tubing. In this example the proximal end of the hypodermic tubing does not need to be sharpened or reshaped. As illustrated, in FIG. 13, a disclosed electrode 10 is covered with an acrylic molding 52 thereby encapsulating the electrode 10 and then inserts snugly into the tubing 40. Although fabrication techniques are disclosed herein, resulting in varying configurations of mounted electrodes, other techniques and configurations are possible. In general, an electrode can be combined or mounted with a hypodermic tube to support, position, protect, and/or facilitate further connections to the electrode. Combining or mounting can include co-locating or attaching (e.g. using any appropriate adhesive glue, cement, solder, tying, wrapping, clamping, crimping, or any other form of co-locating, physically associating, positioning, fixing or attaching known to one of ordinary skill in the art).
1. Attachment of an Electrode to a Hypodermic Tube in a Non-Dura-Piercing Configuration

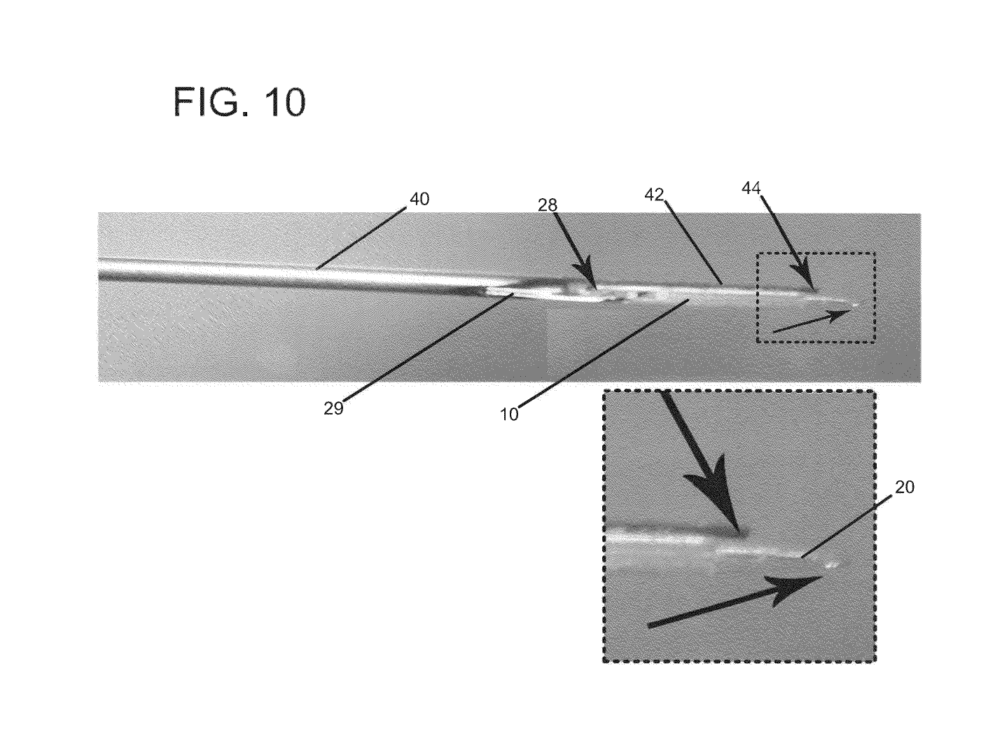

FIG. 10 shows an example of one fabrication technique, in which an electrode can be assembled into a hypodermic tube 40.

Hypodermic tubing can be made of various materials, including various stainless steel alloys, other metal alloys, ceramic and plastic. In some embodiments, the material is corrosion resistant, resistant to solvents, and readily sterilized. In some embodiments the material can be a conductor and enable an electrical connection, such as to ground. In other embodiments, the material can be a non-conductor. In some embodiments, the material provides rigidity or stiffness as well as toughness to enable insertion without bending or breaking, as described below. In certain configurations as described below, the material preferably provides hardness sufficient to be sharpened and to remain sharp.

The tubing dimensions can include various diameters and lengths. Diameters can range between inner diameters of 0.004 inches to 0.05 inches (roughly between 0.1 and 1.27 millimeter(mm)) and outer diameters of 0.005 inches to 0.06 inches (roughly between 0.12 and 1.5 mm). In some embodiments, tubing gauge ranges from 20 to 38, such as from 20 to 28, including 20, 21, 22, 23, 24, 25, 26, 27 or 28.

Tubing length can range from 0.5 inch to 24 inches (roughly between 1.0 and 20 centimeters(cm)), such as from 2 inches to 6 inches (roughly between 6 and 15 cm). In some examples, tubing length is 4 inches (roughly 10 cm).

Toward the proximal end, the tubing can be cut at a steep angle, removing a length of the tubing wall on one side of the tubing only, and leaving the opposing tubing wall intact to form a shelf 42. The tip 44 of the shelf is then the extreme proximal portion of the tubing. Formation of the shelf exposes a corresponding length of the interior of the tubing, the shelf being formed by the exposed interior wall of the tubing. An electrode 10 can be secured into this open portion of the tubing interior by attaching the electrode to the shelf 42.

The position along the length of the shelf at which the electrode is attached can be varied, this gives rise to at least two possible configurations. The first configuration more completely exposes the electrode and thus facilitates cleaning of the electrode and reduces overall mounted assembly diameter. The second exposes the electrode less but better protects the less exposed electrode when the electrode is inserted into a brain. For example, when the electrode is inserted into a mammalian brain and must pierce the mammalian dura, the dura being the outermost and toughest layer of the meninges of the brain. For these reasons, the first configuration is called “non-dura-piercing” and the second configuration is called “dura-piercing.”

The example of FIG. 10 shows the “non-dura-piercing” configuration. In this configuration, a position is selected along the length of the shelf 42 for attachment of the electrode 10 so that a proximal portion of the electrode, for example the electrode tip 20, extends beyond the shelf 42. Thus, while the rest of the electrode is attached to the shelf, the electrode tip is unattached on all sides and more readily accessible for cleaning.

For fabricating a non-dura-piercing configuration, a hypodermic tubing is selected, cut and shaped to a desired length as shown FIG. 10. The tubing gauge is preferably large enough to fit the lead wires with adjacent bonding material inside the lumen of the tubing. Tubing sizes are described above. The tubing can be de-burred using fine grit sandpaper. As shown in FIG. 10, the distal end of the electrical lead wires 29 can be inserted into the cut (proximal) end of the tubing 40 so that the wires are fed into and pass entirely through the lumen of the tubing. In an example, the electrode 10 is also inserted partially into the lumen of the cut end of the tubing so that the bonding pads 28 are inside and protected by the tubing. In another example, the electrode is closer toward the proximal end of the tubing where it sits within the half lumen of the tubing but not within the uncut/whole lumen of the tubing. In an example, the electrode tip 20 is aligned with the tubing shelf 42 so that the electrode tip extends beyond the end of the shelf 44. In an example, the electrode is seated within and glued to or otherwise mounted onto the half-lumen or shelf 42 of the tubing, as described previously.

Insulation can be removed from the distal end of the electrical lead wires 29. In an example, the insulation is polyimide and can be removed by exposing the distal ends of the wires to acid. The wires can then be fixed in electrical contact with an electrical connector 48 or directly to recording equipment. In an example, the wires are soldered to separate connections on an electrical connector 48, and the connector is attached to the mounted electrode assembly by any desirable means.
2. Attachment of an Electrode to Hypodermic Tube in a Dura-Piercing Configuration

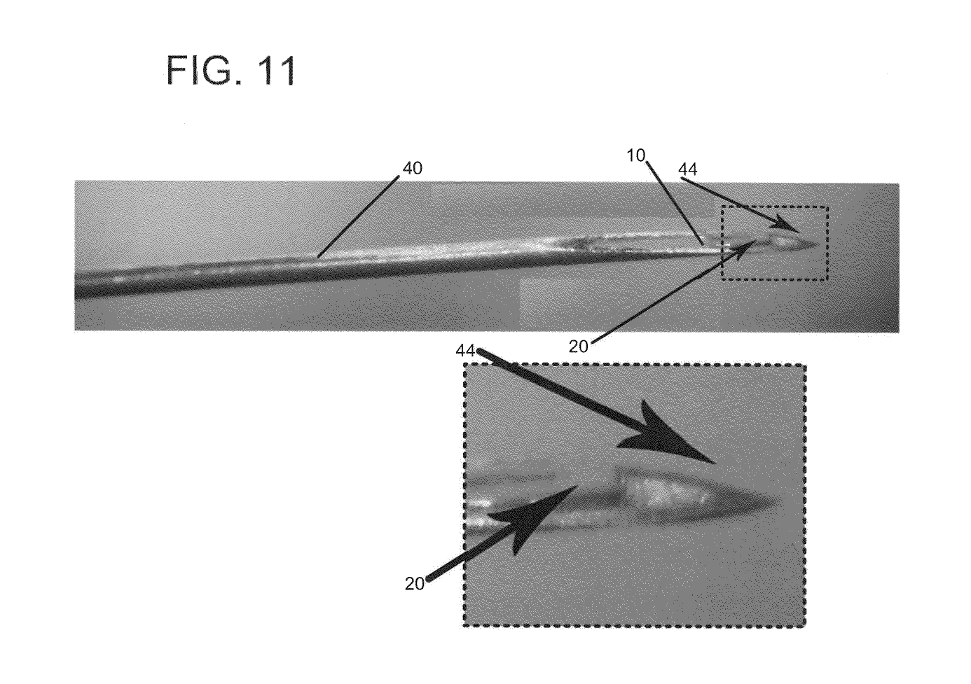

In contrast to the above configuration for attaching an electrode to the hypodermic tubing, an alternative configuration can be used, for example when it is desirable for the electrode to pass through the dura into the brain of a primate. In this alternate configuration as shown in FIG. 11, an electrode 10 is attached to a hypodermic tube 40 so that the all of the electrode, including the electrode tip 20, is positioned distal to the extreme proximal tip 44 of the hypodermic tubing. This positioning allows the proximal tip 44 of the hypodermic tubing to make first contact with and pass first through dura and into the brain.

The proximal tip 44 of the hypodermic tubing 40 can be sharpened to facilitate insertion. Insertion can be especially challenging when preceded at the same location of the dura by previous insertions. In this case, the dura can become thickened through the formation of granulation tissue, which often occurs as a part of normal wound healing. Granulation tissue begins to form upon the exposure of the dura (following implantation of a recording chamber system) even without tissue penetration. Penetration of the dura substantially speeds the development of granulation tissue, although the rate of growth varies between individuals. Attempting to pass through this “granulated dura” usually destroys fragile recording devices. Thickened dura varies from animal to animal and is identifiable by means known to one of ordinary skill in the art. In this case, the shaping, sharpening, and use of steel regarding the hypodermic tubing and the shielding of the electrode away from the tubing's tip and within the half lumen of the exposed interior of the tubing can all be factors enabling and facilitating insertion through the dura, especially thickened dura. Similarly, in some examples insertion alone is insufficient, and proper alignment of the electrode within the brain is relevant. In such examples, these same characteristics of the dura-piercing configuration can facilitate proper alignment.

For the dura-piercing configuration, the electrode can be attached to the hypodermic tubing by selecting a hypodermic tubing, cutting and then shaping the tubing to a desired length with a cutting tip as shown FIG. 11. The tubing gauge is preferably large enough to fit the electrode and lead wires with adjacent bonding material inside the lumen of the tubing. Tubing sizes are described above. The tubing can be de-burred using fine grit sandpaper. As shown in FIG. 10, the distal end of the electrical lead wires 29 can be inserted into the cut (proximal) end of the tubing 40 so that the wires are fed into and pass entirely through the lumen of the tubing. In an example, the electrode 10 is also inserted partially into the lumen of the cut end of the tubing so that the bonding pads 28 are inside and protected by the tubing. In an example, the entire length of the electrode, including the tip, is seated within the half-lumen of the cut end of the tubing, as described previously. Distal portions of the electrode and possibly also the electrode tip are then glued or otherwise mounted into the lumen of the tubing so that no portion of the electrode or the electrode tip extends near the proximal end of the tubing (see FIG. 11).

Insulation can be removed from the distal end of the electrical lead wires 29. In an example, the insulation is polyimide and can be removed by exposing the distal ends of the wires to acid. The wires can then be fixed in electrical contact with an electrical connector 48 or directly to recording equipment. In an example, the wires are soldered to separate connections on an electrical connector 48, and the connector is attached to the mounted electrode assembly by any desirable means.

C. Completion of Mounted Electrode

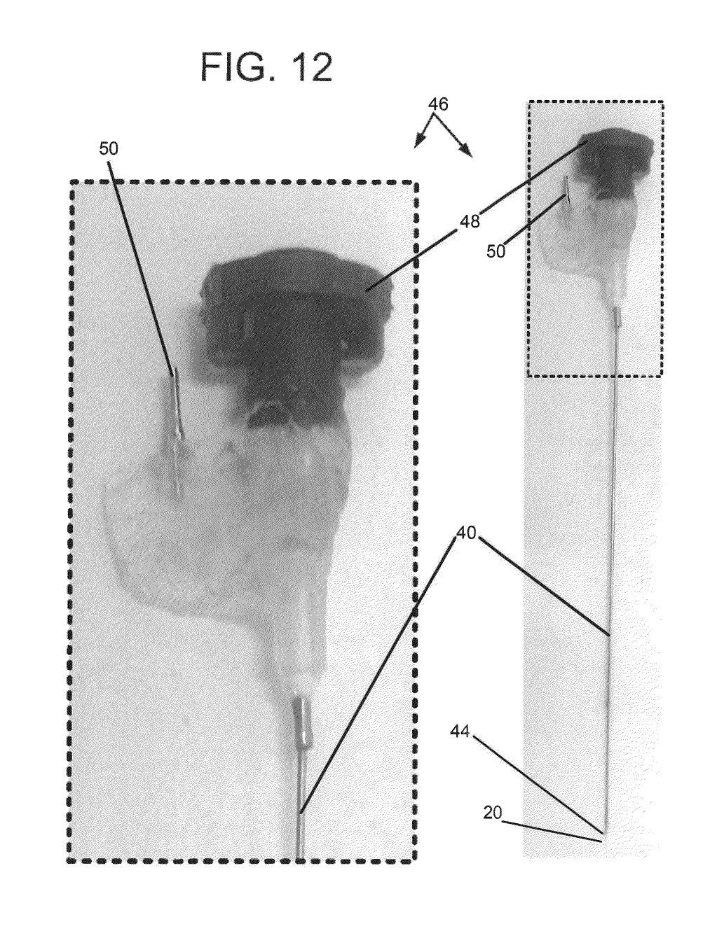

As shown in FIG. 12, after attachment of an electrode 10 to a hypodermic tube 40, next the mounted electrode 46 is completed by attaching other mounting hardware that is portable with the hypodermic tubing and electrode. FIG. 12 shows a dura-piercing configuration, but this final assembly of the mounted electrode can be equally applicable to a non-dura-piercing configuration. In the example shown, a 1× mini-circular plastic connector (CPC) 48 (compatible with the Quanteon electrochemistry potentiostat) and 1× gold pin 50 (compatible with the Plexon electrophysiology amplifier system) also have been attached to the mounted electrode at the distal end of the hypodermic tubing. The connector 48 and pin 50 are soldered to the electrical lead wires 29 (FIG. 10) leading to the bonding pads 28 (FIG. 10) of the electrode. Connector selection is determined by compatibility with the third party hardware/software systems used for making the electrochemical and/or electrophysiological recordings (e.g. Quanteon or BASi for electrochemistry and Plexon or Tucker-Davis for electrophysiology).

The completed mounting assembly or mounted electrode can be compatible with cylinder, grid or guide tube systems used in primate recording, such as the chamber and grid system made by Crist instruments. By compatible it is meant that the connectors and/or hypodermic tube size can be used with, connected to, fitted into, manipulated by, controlled by, or otherwise used with the above guide tube systems. Compatibility is determine and ensured by selecting hypodermic tubing sufficiently small to pass through the guide tube or grid in use and cutting that tubing to a length that allows the mounted electrode to pass down the full height of the chamber (which can be 1 inch or more) and on to the desired depth in the primate brain.

D. Cleaning electrode recording sites prior to use.

Prior to use and insertion of the mounted electrode/mounting assembly into a brain, at least the portion to be inserted is cleaned. In some examples, acetone or CitriSolv (Fisher Scientific) is used as a cleaning agent. Other solvents can also be used. Preferably the cleaning agent does not contact the superglue that attaches electrode to the tubing. A non-dura piercing configuration can be immersed in cleaning agent, up to the end of the tubing. Preferably, dura-piercing configurations are not immersed. After cleaning, the mounted electrode including electrode can be tested and calibrated. After testing and calibration, the electrode can be sterilized, by immersion in 70% ethanol for greater than 10 minutes or by exposure to ethylene oxide gas or irradiation. Some configurations will be compatible with heat sterilization including autoclaving, determined by selection of connectors, method of attaching electrode to the mounting apparatus, choice of insulation layer (e.g. SU-8 or polyimide) and any surface modification of the electrode itself (e.g. application of enzymes and other layers required for some electrochemical techniques). After sterilizing, the mounted electrode including electrode are ready for insertion and use. As an example, the non-dura-piercing configuration (FIG. 10) of the mounted electrode can be used with a separate sharpened guide tube (not shown) in order to pass through dura.

The following examples are provided to illustrate certain particular features and/or embodiments. These examples should not be construed to limit the invention to the particular features or embodiments described.

### Example

This example illustrates the ability to record both an electrophysiological and electrochemical tracings from the primary visual cortex by using a disclosed electrode made in accordance with the methods disclosed herein.

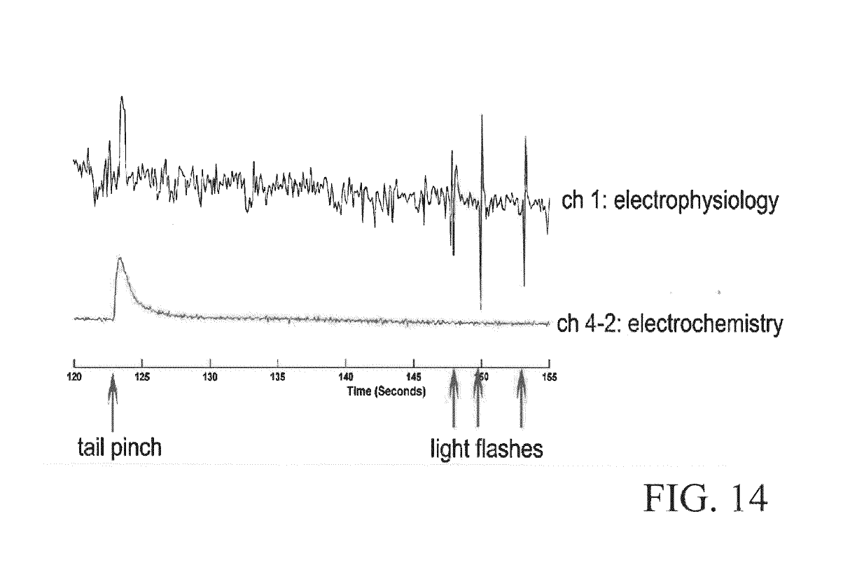

Electrophysiological and electrochemical tracings as shown in FIG. 14 were obtained by positioning an electrode in the primary visual cortex of an awake and freely moving rat. The device was patterned using the masks in the present disclosure. The device was formed on a Coors Superstrate® ceramic wafer and included a 5 nm Ti adhesion layer and a 250 nm Pt recording layer insulated with a 750 micron thick layer of SU-8. After dicing, all four bonding pads had 99% pure Pt wires attached, and those wires had a 1× polyimide insulation layer. The device was assembled into a 27 gauge stainless steel hypodermic tube, in a non-dura piercing configuration. The device was 2 cm in length. The two electrochemistry channels were soldered to a mini-CPC connector and the two electrophysiology channels were soldered to gold pins. The channels referred to as 1 and 3 were electrophysiological channels. The channels referred to as 2 and 4 were electrochemical channels. An enzyme (choline oxidase) was applied to channel 4 to enable the device to detect choline and then electroplate m-phenylendiamine (mPD) was applied onto channels 2 and 4 to block other molecules. Recordings were made by subtracting the signal from channel 2 (no enzyme) from the signal on channel 4 (enzyme coated), giving the current attributable to choline in the tissue. Once enzyme-coated and m-PD plated, the generated electrode was implanted in the primary visual cortex of a rat (under anesthesia) and stabilized using bone cement. A chloride silver wire was implanted under the skin above the left shoulder as a reference for electrochemistry, a bone screw implanted in the skull as a reference for electrophysiology.

After the animal recovered from surgery the recording was made when he was awake and running around in an open field. Electrophysiology was recorded using a Plexon MAP system and electrochemistry using the Quanteon FAST MKII. The electrochemistry system (potentiostat, computer and monitor) were run off an uninterruptible power supply, not mains power. The Plexon was run off mains power.

In the recording, channel 1 of physiology is only shown because 3 is basically a duplication of the recording shown in channel 1. In FIG. 14, time is on the x axis in seconds; physiology (channel 1, LFP recording, bandpass at 0.1-150 Hz); and electrochemistry (channel 4 minus channel 2). At the time point denoted by the arrow, the animal's tail was pinched to induce choline transients in the cortex. At the three time points denoted as light flashes, an LED light was flashed once in the visual field of the animal. Because this is visual cortex, there was a small LFP response to each flash, but no choline response—the animal was habituated to LED flashes so a choline response to this stimulus is not expected. These studies indicate that the disclosed electrodes are effective of recording both electrophysiological and electrochemical activities in the primary visual cortex.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method of making a mounted electrode for recording, comprising:
creating one or more circuits on a wafer;
dicing the wafer to isolate an electrode; and
combining the electrode and a hypodermic tube or molded acrylic, thereby forming an electrode capable of simultaneously measuring electrophysiological activity and a electrochemical environment.

2. The method of claim 1, wherein the one or more circuits comprises at least two circuits, wherein the at least two circuits enable simultaneous electrophysiology and electrochemical recordings.

3. The method of claim 1, wherein the electrode and the hypodermic tube together are capable of piercing thickened dura of a primate brain and inserting the electrode into the primate brain.

4. The method of claim 3, wherein the hypodermic tube has an outer diameter less than or equal to 0.6 mm.

5. The method of claim 4, wherein the electrode has a width less than or equal to 0.4 mm.

6. The method of claim 3, wherein the electrode is compatible with guide systems used in primate recording.

7. The method of claim 3, wherein the electrode can be inserted to any depth within the primate brain.

8. The method of claim 3, wherein the hypodermic tube is at least 1 cm long.

9. An electrode, comprising:
a first electrical circuit enabling electrophysiology recording; and
a second electrical circuit enabling electrochemical recording,
wherein the electrode is capable of piercing thickened primate dura and simultaneously measuring electrophysiological activity and a electrochemical environment within the primate dura.

10. The electrode of claim 9, wherein the electrode is mounted to a hypodermic tube.

11. The method of claim 10, wherein the hypodermic tube has an outer diameter of less than or equal to 0.6 mm.

12. The method of claim 10, wherein the electrode has a width of less than or equal to 0.4 mm.

13. The method of claim 9, wherein the electrode is compatible with guide systems used in primate recording.

14. The method of claim 9, wherein the electrode can be inserted to any depth within the primate brain.

15. The method of claim 10, wherein the hypodermic tube is at least 1 cm long.
